(19) United States
(12) Patent Application Publication
(10) Pub. No.: US 2014/0025024 A1
(43) Pub. Date: Jan. 23, 2014

(54) DISPOSABLE PERSONAL HYGIENE DEVICE

(71) Applicant: Patricia Wallace, Collinsville, IL (US)

(72) Inventor: Patricia Wallace, Collinsville, IL (US)

(21) Appl. No.: 13/554,595

(22) Filed: Jul. 20, 2012

(51) Int. Cl. A61M 35/00

(52) U.S. Cl. 604/310

(57) ABSTRACT

A disposable hygiene device mitt defining a cavity sized and shaped to receive a human appendage, such as a hand or foot. The mitt provides a barrier between the human appendage and unsanitary items or surfaces. A securement member attached to a bottom end of the mitt is configured for securement of the mitt to the human appendage. An emollient is positioned within the cavity for treatment of a medical condition, such as disinfection and cleaning.

### CROSS-REFERENCE TO RELATED APPLICATIONS

Not applicable

### STATEMENT REGARDING FEDERALLY SPONSORED RESEARCH

Not Applicable.

### BACKGROUND

Some patients, such as infants, small children, and especially the elderly, characteristically resist or are unable to cooperate with caregivers during personal care and hygiene. In particular, hands and feet are difficult to clean if a patient is confused or combative. In addition, patients with dementia or Alzheimer's frequently expose themselves to unsanitary surfaces or substances. For example, patients may remove footwear and walk around barefoot, or make content with urine and stool when incontinent.

Therefore, a device for improving personal hygiene of patients is needed.

### DESCRIPTION OF THE DRAWINGS

In the accompanying drawings which form part of the specification:

FIG. 1 is a front view of a disposable personal hygiene device in accordance with the present disclosure;

FIG. 2 is a side view of the disposable personal hygiene device in accordance with the present disclosure; and

FIG. 3 is a perspective end view of the disposable personal hygiene device in accordance with the present disclosure.

Corresponding reference numerals indicate corresponding parts throughout the several figures of the drawings.

### DETAILED DESCRIPTION

The following detailed description illustrates the claimed invention by way of example and not by way of limitation. The description clearly enables one skilled in the art to make and use the disclosure, describes several embodiments, adaptations, variations, alternatives, and uses of the disclosure, including what is presently believed to be the best mode of carrying out the claimed invention. Additionally, it is to be understood that the disclosure is not limited in its application to the details of construction and the arrangements of components set forth in the following description or illustrated in the drawings. The disclosure is capable of other embodiments and of being practiced or being carried out in various ways. Also, it is to be understood that the phraseology and terminology used herein is for the purpose of description and should not be regarded as limiting.

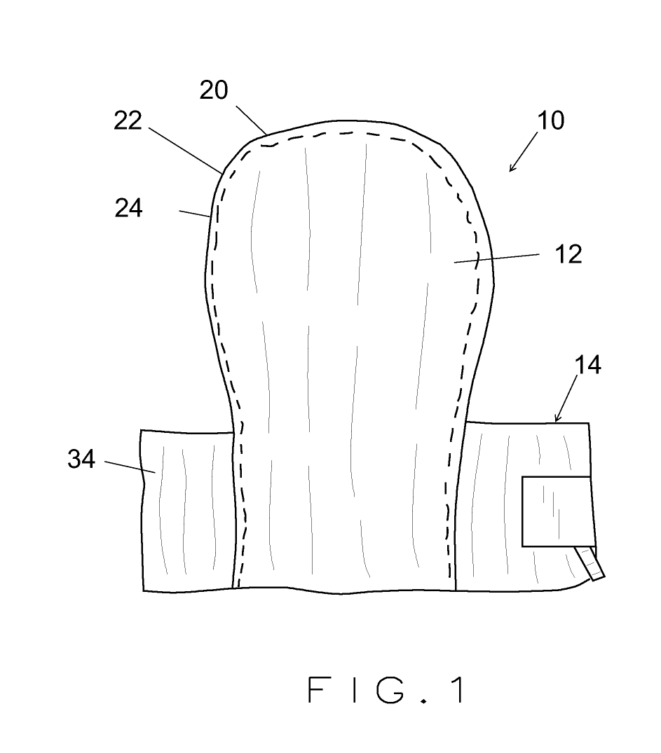

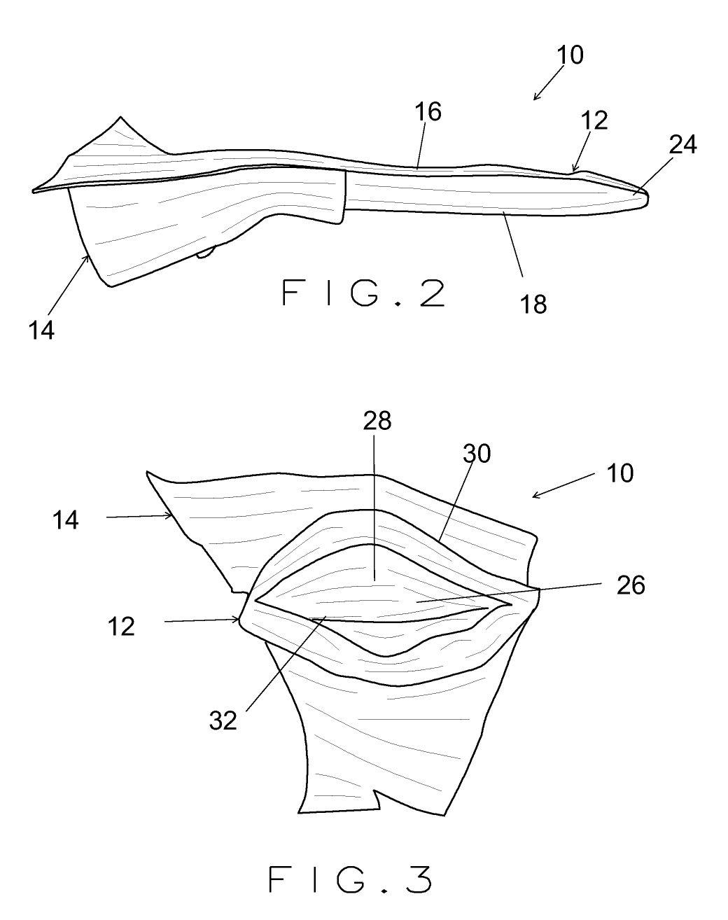

As shown in FIGS. 1-3, an embodiment constructed in accordance with the present disclosure, generally referred to as a disposable personal hygiene device 10, includes a mitt 12 sized and shaped to receive an appendage, such as a generally rounded glove or generally foot-shaped boot, with a single cavity to receive a hand or foot, including all digits (fingers, thumb, toes). A securement member 14 is positioned at a bottom portion of the mitt 12 for removable securement to a person's wrist or arm.

The mitt 12 includes a front panel 16 and a rear panel 18 that are attached along corresponding outer edges 20 and 22 to form a seam 24 and define a cavity 26. The front panel 16 and rear panel 18 are preferably attached with stitching. However, any suitable method can be used, including, but not limited to, adhesive, welding, molding, or staples. Alternatively, the mitt 12 can be manufactured as a unitary glove with a single cavity for receipt of an appendage, rather than attachment of two separate panels.

Both the front panel 16 and the rear panel 18 are preferably made form a multilayer material. In the embodiment of FIGS. 1-3, the inner layer 28 is preferably made of a hygienic absorbent material, such as cotton, gauze, tissue, or other material. The outer layer 30 is preferably made from a medical grade barrier material capable of preventing fluid or bacteria from passing through to the inner layer 28, including, but not limited to, non-woven fabric, polyethylene, polypropylene, Tyvek®, or other material. Those skilled in the art will recognize that the front panel 16 and rear panel 18 can comprise additional layers. For example, additional layers may be added to increase the effectiveness of the fluid and bacterial barrier.

An emollient 32 is applied to the inner layer 28 to treat the user's appendage when inserted in the cavity 26. The emollient 32 can be applied to the inner layer 28 in any suitable manner, such as embedded in the inner layer 28, or in a separate layer on top of the inner layer 28. Any suitable emollient can be used, including, but not limited to petrolatum, lanolin, mineral oil, or dimethicone. In alternate embodiments, other topical medications can be applied within the cavity to address specific medical purposes. For example, an emollient can be used to soften the nails and cuticles of the person's appendage, or include antibiotic properties for cleaning and sanitizing. Preferably, the emollient is activated with warm water so that the personal hygiene device becomes a wash, soak, softener, and comfort device.

The securement member 14 includes a pair of tabs 34 extending generally perpendicularly from the bottom portion of the mitt 12 in opposing directions. The tabs 34 are configured to detachably couple to each other around a person's wrist or ankle for securement of the mitt 12 to the person's appendage, such as with an adhesive bandage, hook and loop device, Velcro®, tape, clasp, or other device.

In operation, a person's appendage inserts into the cavity 26 of the mitt 12. The tabs 34 are wrapped around the person's wrist or ankle for securement of the mitt 12 to the appendage. In this position, the emollient 28 contacts the person's appendage to perform a designated medical treatment soaking, cleansing, disinfecting, softening and comforting. The generally round shape of the mitt 12 rather than a glove with separate cavities for fingers and thumb, limits the function of the appendage. In this way, the person wearing the mitt 12 is less likely to remove, or at least delay the removal of the personal hygiene device 10 because the mitt 12 limits their ability to pinch and grasp. To the extent that patient attempts to remove the mitt 12 the movement may enhance the effectiveness of the emollient 28 by increasing contact with the appendage. In addition, the mitt 12 can function as a sanitary device by providing a barrier between the appendage and any unsanitary or unhygienic substances or surfaces during use. When use of the hygiene device 10 is complete, the securement member 14 is detached and the mitt 12 is removed from the person's appendage. For example, the medical treatment of the emollient may be complete within 3-5 minutes, at which time the mitt 12 is removed. The hygiene device 10 can be disposed in the trash or in accordance with medical waste procedures. Any remaining emollient on the person's hands can be wiped off or dried with a towel.

Changes can be made in the above constructions without departing from the scope of the disclosure, it is intended that all matter contained in the above description or shown in the accompanying drawings shall be interpreted as illustrative and not in a limiting sense.

1. A personal hygiene device, comprising:
a mitt defining a cavity sized and shaped to receive a human appendage, the mitt capable of preventing passage of fluid and bacteria therethrough;
a securement member attached to a bottom end of the mitt and being configured for securement of the mitt to the human appendage;
an emollient positioned within the cavity for treatment of a medical condition; and
wherein the mitt is configured to provide a barrier between the human appendage and external items or surfaces.

2. The hygiene device of claim 1 wherein the mitt comprises:
an inner layer; and
an outer layer capable of preventing passage of fluid and bacteria therethrough.

3. The hygiene device of claim 1 wherein the mitt comprises:
a first panel; and
a second panel attached to the first panel along a seam.

4. The hygiene device of claim 1 wherein the securement device comprises a pair of tabs extending from the bottom portion of the mitt, the tabs being capable of detachably coupling to each other.

5. The hygiene device of claim 1, wherein the mitt is configured to limit the function of the human appendage to prevent interference with medical equipment.

6. (canceled)

7. A disposable hygiene device, comprising:
a mitt defining a cavity sized and shaped to receive a human appendage, the mitt configured to limit the function of the human appendage, the mitt capable of preventing passage of fluid and bacteria therethrough, the mitt configured to provide a barrier between the human appendage and external items or surfaces;
a securement member attached to a bottom end of the mitt and being configured for securement of the mitt to the human appendage; and
an emollient positioned within the cavity for treatment of a medical condition.

8. The hygiene device of claim 7 wherein the mitt comprises:
an inner layer; and
an outer layer capable of preventing passage of fluid and bacteria therethrough.

9. The hygiene device of claim 7 wherein the mitt comprises:
a first panel; and
a second panel attached to the first panel along a seam.

10. The hygiene device of claim 7 wherein the securement device comprises a pair of tabs extending from the bottom portion of the mitt, the tabs being capable of detachably coupling to each other.

11. A disposable hygiene device, comprising:
a mitt defining a cavity sized and shaped to receive a human appendage, the mitt configured to limit the function of the human appendage, the mitt capable of preventing passage of fluid and bacteria therethrough, the mitt configured to provide a barrier between the human appendage and external items or surfaces;
a means for securing the mitt to the human appendage; and
an emollient positioned within the cavity for treatment of a medical condition.

12. The disposable hygiene device of claim 11 wherein the means for securing comprises a pair of tabs extending from the bottom portion of the mitt, the tabs being capable of detachably coupling to each other.
